(19) United States
(12) Patent Application Publication
(10) Pub. No.: US 2013/0066302 A1
(43) Pub. Date: Mar. 14, 2013

(54) INTRAUTERINE ELECTRONIC CAPSULE FOR ADMINISTERING A SUBSTANCE

(71) Applicants: Olaf Hermann WEINER, Briarcliff Manor, NY (US); Jeff SHIMIZU, Briarcliff Manor, NY (US); Hans ZOU, Briarcliff Manor, NY (US); Ventzeslav Petrov IORDANOV, Briarcliff Manor, NY (US); Johan Frederik DIJKSMAN, Briarcliff Manor, NY (US)

(72) Inventors: Olaf Hermann WEINER, Briarcliff Manor, NY (US); Jeff SHIMIZU, Briarcliff Manor, NY (US); Hans ZOU, Briarcliff Manor, NY (US); Ventzeslav Petrov IORDANOV, Briarcliff Manor, NY (US); Johan Frederik DIJKSMAN, Briarcliff Manor, NY (US)

(73) Assignee: MEDIMETRICS PERSONALIZED DRUG DELIVERY B.V., Eindhoven (NL)

(21) Appl. No.: 13/498,835

(22) PCT Filed: Sep. 21, 2010
(86) PCT No.: PCT/IB10/54269

§ 371 Date: Nov. 13, 2012

Related U.S. Application Data
(60) Provisional application No. 61/246,802, filed on Sep. 29, 2009.

(51) Int. Cl. A61B 17/425; A61M 37/00

(52) U.S. Cl. 604/891.1

(57) ABSTRACT

An electronic capsule (200) features a reservoir (204), and is configured for placement within a reproductive organ, such as the uterus or cervix. While located therein, the capsule dispenses (208), from the reservoir, a substance, such as a reproductive hormone, semen, acidic buffer, fertility drug or other drug, effectively administered from within the organ. In some embodiments, the capsule has on-board sensors (238, 244) and control circuitry in wireless communication with an external processor functioning automatically or guided by a clinician or user, for decisions and timing in administering the sub stance.

### FIELD OF THE INVENTION

The present invention is directed to an electronic capsule for placement in a body organ and, more particularly, for placement in a reproductive organ and for dispensing a substance from within the organ.

### BACKGROUND OF THE INVENTION

Infertility, or the inability of a couple to conceive a child, affects about 7.3 million women and their partners in the U.S.—about 12% of the reproductive-age population (Source: National Survey of Family Growth, CDC 2002). Treatment approaches run a wide range from monitoring body signs for ovulation to assisted reproductive technology such as in-vitro fertilization. Even with various treatment approaches many couples still have difficulty in bearing children. Unexplained (idiopathic) infertility is a diagnosis made by exclusion in couples who have not conceived and in whom standard investigations have not detected any abnormality. It accounts for about 40% of female infertility and 8-28% of infertility in couples. Later stage treatments can be very costly. A typical cycle of in-vitro fertilization costs about $12400 and several cycles may be necessary.

U.S. Pat. No. 7,044,911 to Drinan et al. mentions a sensor-bearing capsule for placement in the uterus or vagina. The sensor could detect a change in temperature indicative of ovulation, thereby indicating a time at which sexual intercourse is most likely to lead to fertilization. The capsule would electronically send out an alert signal to the user. Hormonal levels that indicate such a time could also be detected.

Although the alert is helpful, especially to those of degraded fertility, for achieving pregnancy or achieving it quickly, many other factors and contingencies are unmet or unresolved.

### SUMMARY OF THE INVENTION

What is proposed herein is intended to address one or more of the shortcomings of the prior art.

Infertility problems often do not have clear causes and a variety of remedies are tried.

It is generally a goal to choose the least invasive, burdensome, and costly solution that is effective.

One approach is monitoring and predicting the time of ovulation. Alternatively, monitoring urine samples for hormone levels is more accurate.

Automating these processes makes them more convenient for the patient and less subject to error. Also, data can be easily and accurately shared with one's doctor.

Often, however, there are problems with ovulation, with drugs being therefore given to induce ovulation. Overstimulation that causes the release of multiple eggs raises the probability of multiple pregnancies. Multiple pregnancies are an unwanted outcome as they are strongly associated with greater risk for development problems, pregnancy difficulty to the mother, and higher costs.

Administration of the drugs must be timed to the patient's cycle, which does require some monitoring and scheduling. Many of the hormonal drugs are given by injection such as human chorionic gonadotropin (hCG), human menopausal gonadotropin (hMG), and gonadotropin releasing hormone (GnRH).

Injection is an inconvenient route of administration requiring office visits or training for self administration.

Injections also have higher requirements for purity and sterility.

Most drugs used in treatments are systemically available. Systemic drugs are generally more prone to side-effects as opposed to local or topical delivery.

Drug stimulated ovulation is often practiced along with artificial intra-uterine insemination (IUI). IUI requires an office visit and must be conducted according to the time frame of the patient's ovulation schedule.

Lastly if these approaches fail, in-vitro fertilization is recommended. This procedure can be quite costly, invasive, and demanding of the patient.

In one aspect of the present invention, an electronic capsule features a reservoir, and is configured for placement within a reproductive organ. While located therein, the capsule dispenses, from the reservoir, a substance effectively administered from within the organ.

In another aspect, the capsule has a biosensor for measuring the level of a hormone, and is further configured for the dispensing based upon the measured level.

In a sub-aspect, the measuring is for one or more of estradiol, luteinizing hormone and follicle-stimulating hormone.

As a further aspect, the capsule is so configured with the organ being a uterus.

In a sub-aspect of the above, the capsule has a string extending from the capsule into a cervix.

In yet another aspect, the capsule is so configured with the organ being a cervix.

In a different aspect, the dispensing is timed to the reproductive cycle of the host of the capsule.

In some versions, the capsule includes a pH sensor, and is configured such that the dispensing is responsive to output of the sensor.

In a particular aspect, the capsule is so configured with the substance comprising a fluid carrying sperm.

In a general aspect, the capsule is configured for sensing, from an ambient environment, a time for the effective administering, and for the dispensing of the substance at that time.

In one other aspect, the capsule is so configured with the substance comprising progesterone.

In an alternative aspect, the capsule is so configured with the substance comprising a drug.

In a yet further aspect, the capsule is further configured for the dispensing for managing hormone levels for managing endometriosis.

In one particular aspect, the capsule is dimensioned so that it is not expelled by a body comprising the organ.

In a particular sub-aspect, the capsule is so dimensioned without need for an outer carrier for preventing the expulsion.

In an alternative version, a dispensing hole of the reservoir has a removable plug of oil.

As a specific aspect, the capsule includes a motor having a threaded shaft supported on a transverse plate to reduce friction.

In a further alternative aspect, a motor compartment of the capsule has, for pressure equalization, a hole to an ambient environment of the capsule.

A method, as proposed herein, includes, in yet an additional aspect, inserting within a reproductive organ an electronic capsule comprising a reservoir. The capsule is configured for placement within the organ. The method further entails, while the capsule is located within said organ, dispensing, from the reservoir, a substance effectively administered from within the organ.

In a sub-aspect, the dispensing involves dispensing an acidic buffer in response to a pH reading.

In an additional sub-aspect, the dispensing involves dispensing a substance to induce ovulation.

In yet one further aspect, a computer software product for operating an electronic capsule having a reservoir and located within a reproductive organ includes a computer readable storage medium embodying a computer program. The program includes instructions executable by a processor to, while the capsule is located within said organ, dispense, from the reservoir, a substance effectively administered from within the organ.

In yet another embodiment, an article of manufacture includes a machine-accessible medium having instructions encoded thereon for enabling a processor to, while an electronic capsule having a reservoir is located within a reproductive organ, dispense, from the reservoir, a substance effectively administered from within the organ.

Details of the novel, electronic, intrauterine, substance-administering capsule, or intelligent iPill, are set forth further below, with the aid of the following drawings, which are not drawn to scale.

### BRIEF DESCRIPTION OF THE DRAWINGS

FIGS. 1A, 1B are schematic diagrams exemplary of how the electronic capsule is placed in a reproductive organ;

FIG. 2 is a schematic diagram of side and bottom views of the electronic capsule;

FIG. 3 is a graph of hormone levels over time during a menstrual cycle, showing, by example, when the dispensing mechanism may be activated; and

FIGS. 4A, 4B are flow charts showing the status and activity of the electronic capsule.

### DETAILED DESCRIPTION OF EMBODIMENTS

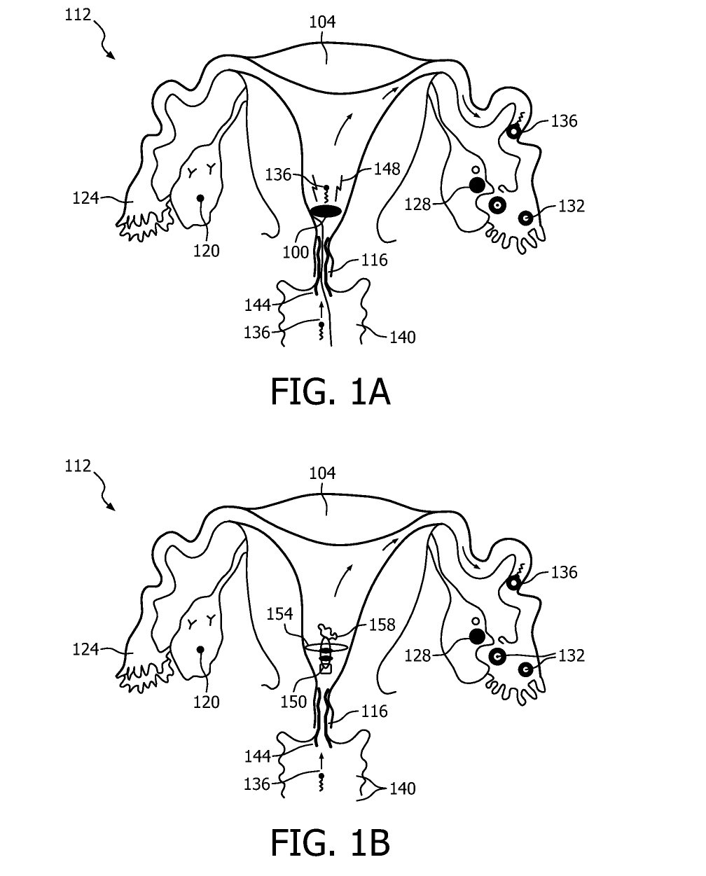

FIG. 1A shows an electronic capsule 100 that has been placed within a uterus 104.

A string 108 is optionally attached for removing the capsule 100 after a mammalian subject or patient 112, i.e., the host of the capsule, becomes pregnant. The string extends down into the cervix 116 and may extend further such as by two inches so as to be easily graspable with the fingers. Also shown are the ovaries 120 where eggs are produced, and the Fallopian tubes 124 in which fertilization occurs. An egg 128 released, or “ovulated,” is shown later as a fertilized egg 132 in a location typical for fertilization. A sperm 136 travels from the vagina 140 up past the cervical mucus 144 and into the uterus 104. The sperm 136 that succeeds in fertilizing the egg 132 travels through a Fallopian tube 124 to encounter and fertilize the egg. Typically, the capsule 100 is, as indicated by the zigzags 148, electronically in communication with a remotely located processor which carries out much of the computation and has access to externally available information. The capsule 100 is sized and shaped, i.e., dimensioned, to prevent expulsion by the body, as through the cervix 116 and out through the vagina 140.

The capsule 150 in FIG. 1B has itself been placed in a larger carrier 154. The capsule 150 is shown dispensing a substance 158, such as a hormone, an acidic buffer, a drug or semen.

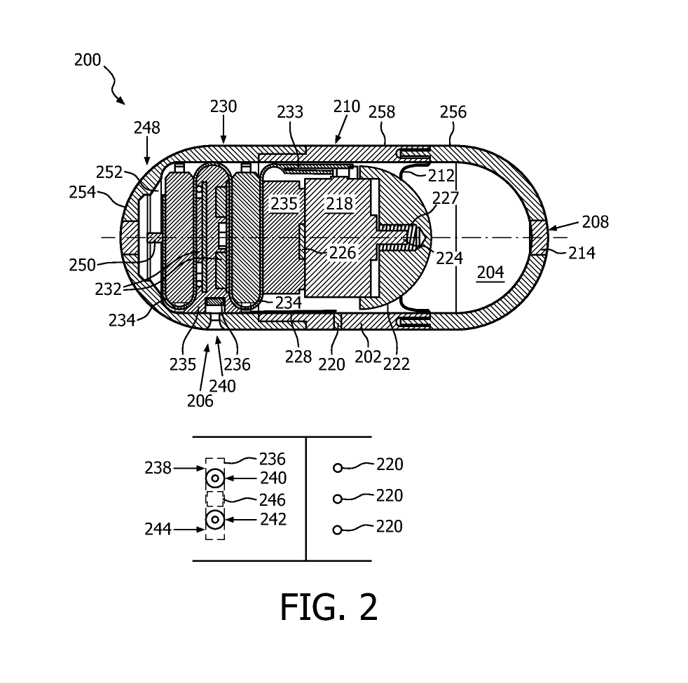

FIG. 2 depicts, by illustrative and non-limitative example, an electronic capsule 200 in accordance with what is proposed herein. The upper part of FIG. 2 portrays a side view of the capsule 200, and the lower part portrays a bottom view, i.e., what would be visible if we were to revolve the capsule 90° in the up direction.

The capsule consists of four distinct compartments within a housing 202 made of bio-compatible material.

A reservoir 204, for storing the substance 158 to be administered, communicates to the direct, ambient environment 206 of the capsule 200 through a dispensing hole 208. This compartment 204 is separated from a motor compartment 210 by a flexible, rolling sock seal 212. The material of the seal 212 may be a polymer based laminate like a pharmaceutical grade Polyethylene/Polychlorotrifluoroethylene (PE/PCTFE) flexible film. To a stud or eyelet 214 protruding from the inner circumference of the dispensing hole 208 the string 108 may be fastened.

The motor compartment 210 housing a stepper motor 218 communicates to the direct, ambient environment 206 through a number of small sized holes 220 in order to keep the pressure inside the capsule 200 exactly equal to the pressure outside the capsule. The stepper motor 218 is loosely fixed. In particular, when a piston 222 moves forward, the threaded shaft 224 supports itself on a small, transverse, metal plate 226 so as to stay fixed in its axial direction. This is done to reduce friction in case the piston 222 is loaded. The spherical shape of the piston 222 along the rolling sock seal 212 widely distributes pressure to, likewise, reduce friction. A threaded insert 227 of the piston 222 engages the threaded shaft 224 so that revolving of the shaft drives the insert forward and therefore the piston which is fixed to the insert. A start-up coil 228 for connecting a switch to the battery is included.

In an electronics compartment 230, all of the electronic components 232, including, among other components, batteries, control and data storage, timing circuitry (not shown) and one or more antennas 233, are mounted on a flex foil 234, which serves as a printed circuit board. The flex foil 234 is made of a polymide substrate such as Kapton™. The components 232 can be attached by soldering. The flex foil integration of components 232, which advantageously reduces the number of components, is described in more detail in commonly assigned International Patent Publication Number WO 2008/062333A2, entitled “Ingestible Electronic Capsule and In Vivo Drug Delivery or Diagnostic System,” to Dijksman et al., the entire disclosure of which is hereby incorporated herein by reference.

After mounting of the components 232, the flex foil 234 is folded, placed in the housing 202 and potted, in mouldable resin 235 for instance, such that content of the ambient environment 206 cannot reach the electronics 232. A measuring surface 236 of an ion-selective field effect transistor (ISFET) 238 directly communicates with the environment 206. The ISFET is usable, for example, to measure pH levels. The communication is by means of an orifice 240 in the housing 202. Another orifice 242 exists for a biosensor 244 for measuring hormone levels. A channel 246 connects the two orifices 240, 242. Motion of a biorecognition element (not shown) of the biosensor 244 and the presence of the channel 246 afford flow of fluid from the ambient environment 206 past the element and past the ISFET measuring surface 236. The biorecognition element can be moved electrically or electromagnetically. A suitable biosensor is disclosed in commonly assigned U.S. Patent Publication 2008/0311679, entitled “Biosensor Device,” to Den Toonder et al., the entire disclosure of which is hereby incorporated herein by reference.

A reference electrode compartment 248 includes a reference electrode 250 for the ISFET 238. The reference electrode 250 consists of a short silver wire covered with silver chloride, and is placed in a gel 252 with 4.5 mol/litre KCl. A fit window 254 seals the gel 252 inside and affords the means by which the reference electrode 250 communicates ionically with the outside of the capsule 200.

For a long shelf life, the electronic control circuitry is completely decoupled from the battery, or other power source, to prevent a small leakage current from expending the power in the battery. A wake-up circuit may be used to activate the coupling. The circuit is powered from outside by inductive radiation that is received by the start-up coil 228.

The housing 202 includes a first part 256 and a second part 258. The first part 256 is secured to the second part 258. The rolling sock seal 212 is clamped between the first part 256 and the second part 258, thereby securely sealing off the motor 212 from the substance 158 to be dispensed. As seen in FIG. 2, the first and second parts 256, 258 are joined in a protrusion/recess configuration. In this way the sealing length of the rolling sock seal 212 is enlarged, which increases the sealing strength of the seal and hence the reliability of the device 200. The sealing properties can be further enhanced by using biocompatible adhesives for the adhesion of the seal 212 in between the protrusion and joining recess. Alternatively, ultrasonic frictional heating or laser welding may be applied.

The housing 202 may be fabricated from a biologically safe polymeric material such as, for example, polytetrafluoroethylene, polypropylene, polyethylene, acrylics and the like. The housing 220 can be manufactured from materials used to fabricate implantable devices, including pacemaker leads and cardiac prosthesis devices, such as artificial hearts, heart valves, intra-aortic balloons, and ventricular assist devices. Optionally, a proper coating is applied to the surface of the device to ensure biocompatibility.

In operation, the piston 222 moves forward, pressing against the flexible, rolling sock seal 212. This causes displacement that forces the substance 158 through the dispensing hole 208. The amount dispensed can be precisely controlled by the stepping action of the stepper motor 218. For simplicity of fabrication and operation the dispensing hole 208 is simply an open window. That is, no mechanical valve is used to separate the medication compartment from the outside environment. To prevent unwanted release of drug, a removable plug, e.g., oil droplet, can be applied to the open window. The shape and size of the window can be chosen to reduce undesired diffusion to a satisfactory level.

Use of the piston 222 to create pressure is safer than using gas. Also, the piston 222 need not conform to tight tolerances in order to hermetically seal the reservoir 204; instead, the rolling sock seal 212 serves that function.

According to the instant proposal, measurement, monitoring, and analysis are used to determine the time point in the reproductive cycle of the patient 112. Drug delivery is used to enhance the cycle and conditions for successful conception. Timing of the drug delivery is controlled by a program internal to the implanted device 200 along with data and analysis communicated between the device and outside equipment.

The capsule 200 is in wireless contact with a receiver system outside the body 112, as by a radiofrequency (RF) link. The receiver system can record data reported by the capsule 200 such as measurements from the sensors 238, 244. Other sensors, such as a temperature sensor, may be on-board the capsule 200. Likewise, only one or fewer than all of such sensors may be on-board. These measurements can give information as to the state of the patient 112. Analysis of the data by computer program or observer can be used as intelligent input to take an action such as delivery of the drug 158 from the same capsule 200.

Monitoring of hormone levels, for example hCG level, other chemical markers, or examination for example by ultrasound may be used to determine when successful embryo implantation occurs and thus when the capsule 200 should be removed. Further considering the device is intended to reside for a fixed period of one to several months, the material of the carrier may be such that it erodes or becomes less rigid over time. This may aid in the removal of the device with a minimum of mechanical trauma to the cervix 116 and uterus 104 during removal.

One function of the device is to monitor in-situ the condition of the patient 112 to determine the time of ovulation. The capsule 200 can contain a temperature sensor for this purpose. The sensor periodically records basal body temperature. The device additionally contains a wireless communication means. For example an RF wireless chip is contained on the device. The device transmits data from the measurements to an external unit. The external unit stores and/or relays measurements to a computing device such as a personal computer. The external unit or personal computer uses the temperature data along with time stamps to chart body temperature much in the way a manual basal body temperature (BBT) chart is used. In this case the measurement, charting, and analysis are automatic, requiring no action from the patient. To provide additional information, the system may be designed to record manual input from the patient to mark the exact time she rises from bed.

While temperature and the BBT chart are good indicators for ovulation it is retrospective and subject to interfering influences. A better indicator is to monitor hormonal levels. Thus the capsule 200 may include the biochemical sensor or biosensor 244 to measure hormonal levels. Candidate analytes include estradiol, luteinizing hormone, and follicle stimulating hormone.

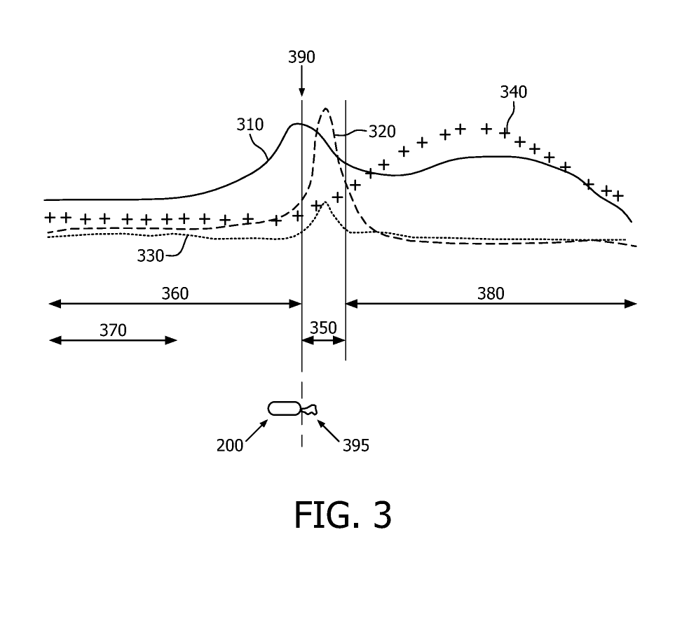

FIG. 3 indicates hormone levels over time during a menstrual cycle, showing, by example, when the dispensing mechanism may be activated. The hormones for which levels are shown are estradiol 310, luteinizing hormone 320, follicle stimulating hormone 330, and progesterone 340. The time periods prior to ovulation 350 are the follicular phase 360 and menstruation 370, and the time period after is the luteal phase 380. As seen from FIG. 3, estradiol 310 is a particularly effective predictor for the commencement of ovulation, although there is a noticeable rise in the other hormones 320, 330, 340.

Levels of estradiol 310 and luteinizing hormone 320 are detectable in the uterine environment, as by means of the biosensor 244 discussed hereinabove. Again measurements are reported to an external unit, recorded, and analyzed. Data analysis at the external unit or computing device will indicate to the patient 112 and/or doctor when the optimal time for conception is. This may be used to plan natural or artificial insemination, which preferably occurs at the commencement 390 of ovulation.

Alternatively or in addition, the capsule 200 may be configured with the pH sensor 238. The pH of the cervix and uterine environment changes with menstrual cycle. An acidic pH is harmful to sperm 136. pH tends to rise near the time of ovulation 350. Thus a monitoring of pH can give information as to whether conditions are favorable for conception. The information can be used for cycle prediction, diagnosing sub-optimal conditions and/or indicating whether corrective actions are desirable such as the release of a substance to raise and buffer the pH.

The substance 158 dispensed from the reservoir 204 may be a drug to stimulate or assist ovulation, such as hormonal, hormonal analogs, or large molecule drugs with local action. Local administration in the uterus is more effective as it is at or near the site of action. Further, hormones or large molecules are typically not orally bioavailable. By releasing the drug from the capsule 200 the need for administration by injection is eliminated. Moreover, since the drug is topically active, the amount of drug reaching systemic circulation shall be much reduced along with drug side-effects. The delivery of the drug is, in some embodiments, appropriately timed to the patient's reproductive cycle. For example, luteinizing hormone 320 is needed at the time of ovulation 350 to induce release of a mature egg 128 from the follicle. Timing and amount of drug delivery is under control of the device electronics 232 that takes action using input from on-board data as well as external data and analysis. External data including timing triggers are communicated wirelessly from the external unit to the capsule 200. The dose and duration of drug delivery can also be adapted to the specifics of the individual 112. As it may be an assist to deficiencies in natural hormonal production, the data from actual body levels can be used to determine the desired delivery dose and duration. This can for example be adapted over the course of delivery which can be over several days.

Alternatively or in addition to promoting proper ovulation, the device may deliver one or more drugs that promote conditions for successful implantation of the fertilized egg 132 to the endometrium, i.e., the cells that line the uterus 104. It is not uncommon for a successful fertilization to end with an unsuccessful implantation. The empty follicle produces progesterone 340 to prepare the endometrium. A defect in this process or proper levels reduces the chance of successful conception. In this case the capsule 200 will deliver progesterone 340. The system is ideally disposed to this mode as progesterone 340 is topically active and is delivered to the uterus 104. Local delivery by the capsule 200 solves problems with poor bioavailability of progesterone 340 by oral delivery or inconvenience of delivery by vaginal enema. Additionally progesterone delivery is preferable to administration of synthetic analogs such as progestin which may carry additional side-effects.

In yet another embodiment, the substance 158 delivered may be a fluid carrying sperm 136, e.g., semen (or seminal fluid containing sperm). This is a kind of artificial intra-uterine insemination. The advantage of delivery by the implanted capsule 200 is that the insemination can happen at the optimal time without the need for an additional office visit necessarily scheduled around the time of ovulation. Here the system monitoring and prediction feature is used to calculate the optimal time for insemination and a trigger signal is delivered wirelessly to the capsule 200. The capsule 200 then releases the semen 395 bearing active sperm 136 at the right time 390.

The system may also find utility in areas other than fertility, i.e., in other gynecology applications, especially where the combination of measurement, monitoring, and drug delivery brings benefits. An example is in treatment for endometriosis. Endometriosis occurs when tissue from the uterine lining attaches to other organs in the body (such as the cervix, vagina, intestines, etc.) and begins to grow. Irritation, pain, and infertility can result. Common treatments include birth control hormones to halt ovulation and the associated growing, shedding, and bleeding of endometrial tissue that makes endometriosis painful. Rather than halting the menstrual cycle altogether, an intelligent, electronic capsule based therapy can be used to deliver medication to manage hormonal levels in order to reduce the amount of tissue swelling that leads to painful endometriosis. Estrogen levels, for example, may be lowered to treat or prevent endometriosis. Obtaining a more natural balance is expected to have fewer side effects that come with conventional drug therapies such as progestin or Danozol™. Side effects include irregular uterine bleeding, weight gain, water retention, breast tenderness, headaches, nausea, and mood changes, particularly depression.

An alternative to placement in the uterus 104 or cervix 116 is to embed or implant the capsule 200. For example implant the capsule 200 subcutaneously. In this embodiment the capsule 200 would be designed to be smaller to ease implantation. Further, depending on a trade off between desired functions and size or difficulties with implants, the capsule 200 may reduce, or be configured with reduced, functions, for example to be strictly a monitor device and system.

Lastly, the capsule 200 may serve as general drug delivery system. In certain applications drugs can be more effectively and conveniently absorbed through the vaginal or uterine tissues. Application is not necessarily restricted to fertility, infertility or gynecology. For example large molecule drugs, proteins, peptides, or other drugs that are poorly absorbed, degraded, or otherwise poorly bioavailable via the intestinal tract may be more effectively delivered through other organs such as the lung, mouth, or eye. Included are the vaginal or uterine routes for women. This area has a thick network of blood vessels that enhances the utility of this route for drug delivery. The capsule 200 can reside in the cervix 116 or uterus 104 and deliver a drug that is absorbed through these organs. This is preferred for example to delivery by injection or infusion. Additionally if the target organs are at or near the uterus 104 then effective therapy may be possible at reduced systemic drug levels, leading to fewer side-effects. Local administration also avoids first pass metabolism as the drug may be directly perfused into the nearby tissues. Among therapy areas where uterine or vaginal delivery may be beneficial are, hormone replacement therapy, urinary incontinence, endometriosis, vaginal microbicides, vaginal vaccination, and pain relief.

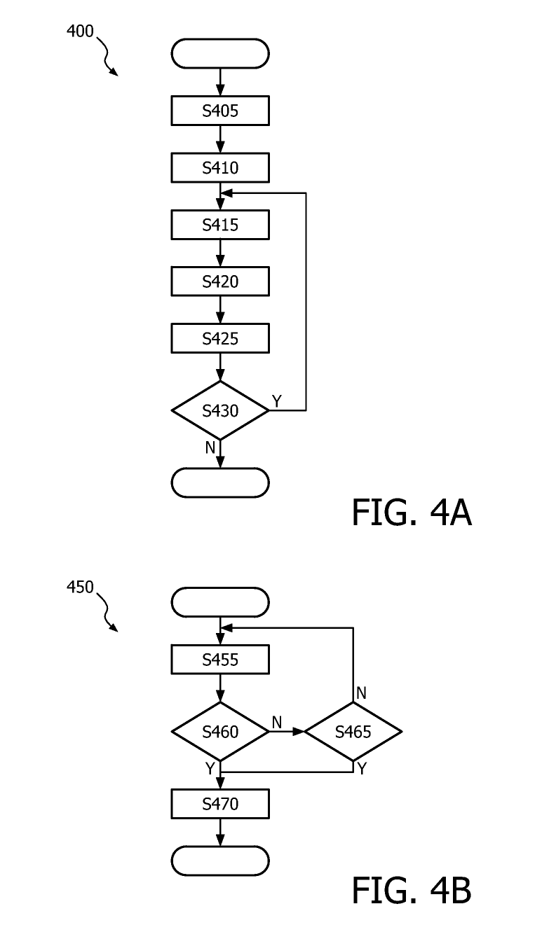

FIGS. 4A, 4B demonstrate conceptually, by example, the status and activity of the electronic capsule 200.

In a dispensing process 400, first the capsule 200 is placed in the reproductive organ (step S405). An external signal wakes up the control circuitry (step S410). These two steps may be performed in reverse order. The capsule 200 then senses the ambient environment 206 and takes readings (step S415). The readings and/or information received from an external source are used by the capsule 200 and/or an external processor to determine one or more dispensing times (step S420). Dispensing of the substance 158 in the reservoir 204 occurs (step S425). If the capsule 200 is still present in the organ (step S430), processing returns to step S415; otherwise, processing ends.

In a monitoring process 450, hormone levels, such as for human chorionic gonadotropin (hCG), are monitored to detect pregnancy (step S455). If the subject 112 is not pregnant (step S460), and the capsule 200 is not expired (step S465), processing returns to step S455. If, on the other hand, the subject 112 is pregnant (step S460) or the capsule 200 is expired (step S465), the capsule 200 is removed from the organ in which it has been placed (step S470). The monitoring process 450 may initially be dormant, and commenced some time before capsule life expiry or after insemination naturally or by means of semen being dispensed from the reservoir 204.

An electronic capsule features a reservoir, and is configured for placement within a reproductive organ, such as the uterus or cervix. While located therein, the capsule dispenses, from the reservoir, a substance, such as a reproductive hormone, semen, acidic buffer, fertility drug or other drug, effectively administered from within the organ. In some embodiments, the capsule has on-board sensors, and control circuitry in wireless communication with an external processor functioning automatically or guided by a clinician or user, for decisions and timing in administering the substance.

The capsule 200 is applied in health-related treatments. In particular, uterine residence, monitoring, and adaptable control make it well-suited for fertility, infertility, or gynecology applications. More generally, the system may be used as a drug delivery system which is preferable to delivery by injection or infusion.

It should be noted that the above-mentioned embodiments illustrate rather than limit the invention, and that those skilled in the art will be able to design many alternative embodiments without departing from the scope of the appended claims. For example, the electronic capsule 200 may be provided with an image sensor and the ability to reorient, or otherwise move, itself to detect or measure the local existence of endometriosis. In the claims, any reference signs placed between parentheses shall not be construed as limiting the claim. Use of the verb “to comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer having a computer readable medium. The mere fact that certain measures are recited in mutually different dependent claims does not indicate that a combination of these measures cannot be used to advantage.

What is claimed is:

1. An electronic capsule (200) comprising a reservoir (204), said capsule configured for placement within a reproductive organ and, while located within said organ, dispensing (S425), from said reservoir, a substance effectively administered from within said organ.

2. The capsule of claim 1, further comprising a biosensor (244) for measuring a level of a hormone, and further configured for said dispensing based upon the measured level.

3. The capsule of claim 2, wherein said measuring is for one or more of estradiol (310), luteinizing hormone (320) and follicle-stimulating hormone (330).

4. The capsule of claim 1, so configured with said organ being a uterus (104).

5. The capsule of claim 4, comprising a string (108) extending from said capsule into a cervix.

6. The capsule of claim 1, so configured with said organ being a cervix (116).

7. The capsule of claim 1, said dispensing being timed to a reproductive cycle of a host (112) of said capsule.

8. The capsule of claim 1, comprising a pH sensor (238), and configured such that said dispensing is responsive to output of said sensor.

9. The capsule of claim 1, so configured with said substance comprising a fluid (395) carrying sperm.

10. The capsule of claim 9, configured for sensing, from an ambient environment, a time (390) for the effective administering, and for said dispensing of said substance at said time.

11. The capsule of claim 1, so configured with said substance comprising progesterone (340).

12. The capsule of claim 1, so configured with said substance (158) comprising a drug.

13. The capsule of claim 1, said capsule being further configured for said dispensing (S425) for managing hormone levels for managing endometriosis.

14. The capsule of claim 1, further dimensioned so that it is not expelled by a body (112) comprising said organ.

15. The capsule of claim 14, so dimensioned without need for an outer carrier (154) for preventing the expulsion.

16. The capsule of claim 1, a dispensing hole (208) of said reservoir having a removable plug of oil.

17. The capsule of claim 1, comprising a motor (218) having a threaded shaft supported on a transverse plate to reduce friction.

18. The capsule of claim 1, comprising a motor compartment with, for pressure equalization, a hole (220) to an ambient environment of said capsule.

19. A method comprising:
inserting (S405) within a reproductive organ an electronic capsule comprising a reservoir, said capsule configured for placement within said organ; and
while said capsule is located within said organ, dispensing (S425), from said reservoir, a substance effectively administered from within said organ.

20. The method of claim 19, wherein said dispensing comprises dispensing an acidic buffer in response to a pH reading (244).

21. The method of claim 19, wherein said dispensing comprises dispensing a substance (158) to induce ovulation.

22. A computer software product for operating an electronic capsule having a reservoir and located within a reproductive organ, said product comprising a computer readable storage medium embodying a computer program that includes instructions executable by a processor to perform a plurality of acts, said plurality comprising the act of:
while said capsule is located within said organ (S405), dispensing, from said reservoir, a substance effectively administered from within said organ.

23. An article of manufacture, comprising a machine-accessible medium having instructions encoded thereon for enabling a processor to, while an electronic capsule having a reservoir is located within a reproductive organ, dispense (S425), from said reservoir, a substance effectively administered from within said organ.
